(19) United States
(12) Patent Application Publication
(10) Pub. No.: US 2011/0092938 A1
(43) Pub. Date: Apr. 21, 2011

(54) DIAPER

(76) Inventor: Yinhai Yang, Tianjin (CN)

(21) Appl. No.: 12/886,315

(22) Filed: Sep. 20, 2010

(30) Foreign Application Priority Data
Oct. 19, 2009 (CN) CN200910070855

(51) Int. Cl. A61F 13/15

(52) U.S. Cl. 604/385.01

(57) ABSTRACT

The present invention relates to a diaper. It belongs in the technical field of hygiene care articles. A diaper capable of protecting belly and navel of infants, characterized in that: a protrusion structure is set in the front of the diaper to cover the belly and navel of infants, wherein the protrusion structure is provided with medicament. The present invention can protect infants' belly and navel to not only prevent infants' belly and navel from wind and cold air, but also take care of and effectively treat diarrhea and abdominal distention. Featuring a simple structure, convenient to wear and remarkable therapeutic effects, the present invention is capable of safety protection and pharmacological care in an economical, practical, comfortable and safe way.

### BACKGROUND OF THE INVENTION

1. Technical Field

The present invention relates to hygiene caring articles, and more especially, to a diaper.

2. Description of Related Art

Currently, the paper diapers available in the market have a linear pattern in the front edge. The diaper is suited to the lower part of the belly of infants without covering the navel. Moreover, neonates are most afraid of catching a cold from an exposed navel (belly). However, in our daily life, an infant's belly is often exposed inadvertently. To avoid this, an abdominal belt is provided in the market to specially protect infants' bellies, but this belt is often thick and is often fastened around the waist of the infant. In this way, the infant may feel hot around the waist, leading to discomfort. Meanwhile, it is troublesome since the protection function can be completed only after dressing several times.

### BRIEF SUMMARY OF THE INVENTION

To solve the problem in the existing technology, the present invention provides a diaper.

The present invention aims at providing a medicated diaper capable of protecting the belly and navel of infants, capable of curing diarrhea and protecting the belly and navel of infants, featuring a simple structure, good air permeability, practical and safe application, convenient and comfort dressing.

The present invention has a medicated protrusion structure in the front of the diaper capable of protecting the belly, wherein this structure is the extending portion of the diaper body, or the folded medicated structure coupled with the diaper, to protect the belly and navel. This medicated structure can be made of pure cotton fabric or regular non-woven fabric, or a composite body of absorbing material, dust-free paper, cast film and other materials, or a single material, or various materials capable of protecting the belly and navel of infants.

The diaper capable of protecting the belly and navel of infants of the present invention adopts the following technical solution:

A diaper capable of protecting the belly and navel of infants, characterized in that, a protrusion structure is set in the front of the diaper to cover the belly and navel of infants, wherein the protrusion structure is provided with caring medicament.

The diaper capable of protecting the belly and navel of infants of the present invention can also adopt the following technical solution:

The diaper capable of protecting the belly and navel of infants, characterized in that, the protrusion structure is the extending portion of the diaper body.

The diaper capable of protecting the belly and navel of infants, characterized in that, the protrusion structure is made of a composite body of the prolonged diaper surface and base film or absorbing material, or a composite body of the prolonged diaper surface and base film plus absorbing material.

The diaper capable of protecting the belly and navel of infants, characterized in that, the protrusion structure is made of pure cotton fabric or regular non-woven fabric.

The diaper capable of protecting the belly and navel of infants, characterized in that, the protrusion structure has a printed or pressed pattern.

The diaper capable of protecting the belly and navel of infants, characterized in that, the protrusion structure and the diaper are two separate parts combined into one structure through connection.

The diaper capable of protecting the belly and navel of infants, characterized in that, the protrusion structure is connected with the diaper by means of thermal sealing, adhesion or suturing.

The diaper capable of protecting the belly and navel of infants, characterized in that, caring medicament is provided inside or outside the protrusion structure, the said medicament is in the form of powder, grain, liquid for spraying, paste or absorbent cotton dipped with liquid medicine.

The diaper capable of protecting the belly and navel of infants, characterized in that, the caring medicament can be one or the mixture of two or three selected from clove, cinnamon and long pepper (piper longum).

The present invention can achieve the following advantages and favorable effects:

The new technical solution adopted by the present invention mainly aims at addressing the problem that the belly is often exposed incidentally in daily life which may cause the infant to have a cold. There is abdominal belt especially used for protecting infant bellies in the market, but this belt is often thick and is often fastened around the waist of the infant. In this way, the infant may feel hot around the waist, leading to a sense of discomfort. Meanwhile, this means is troublesome since the protection function can be completed after dressing several times. The present invention is a diaper capable of realizing belly protection directly. No matter how the infant moves, the protection to the belly always functions well. Furthermore, it is provided with medicament capable of invigorating the spleen, warming middle energizer, dissipating cold, stopping diarrhea, treating diarrhea and abdominal distension. It can effectively take care of infants' bellies. It is a partial protection structure featuring effective treatment for infant belly disease, good air permeability, safety, integrity, convenient and comfort dressing, favorable therapeutic effect.

### BRIEF DESCRIPTION OF THE SEVERAL VIEWS OF THE DRAWINGS

FIG. 1 is the schematic view of the structure for the present invention;

FIG. 2 is the schematic view of the structure bonding diaper body and medicated protrusion structure;

FIG. 3 is the schematic view of the medicated diaper structure;

FIG. 4 is the schematic view of the structure suturing diaper body and medicated protrusion structure;

FIG. 5 is the schematic view of the structure connecting diaper body and medicated protrusion structure in FIG. 1;

FIG. 6 is the schematic view of the structure connecting diaper body and medicated protrusion structure in FIG. 2;

FIG. 7 is the schematic view of the structure connecting diaper body and medicated protrusion structure in FIG. 3.

### DETAILED DESCRIPTION OF THE INVENTION

To further understand the technical contents, features and effects of the present invention, the following embodiments in combination with the drawings are used to detail the invention as below:

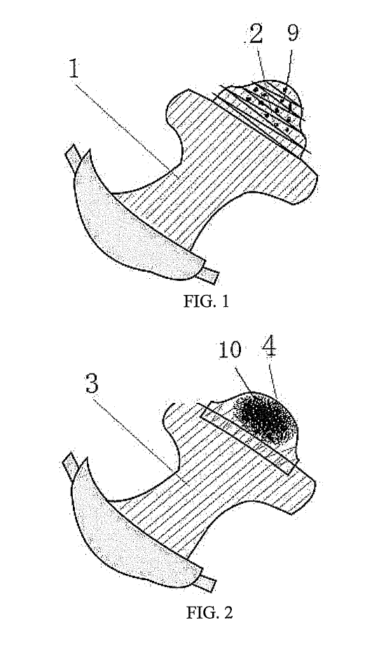

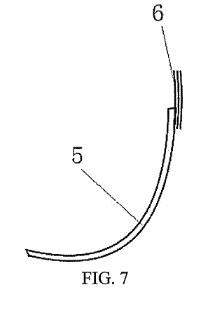

Refer to FIGS. 1, 2, 3, 4, 5, 6 and 7.

### IMPLEMENTATION EXAMPLE 1

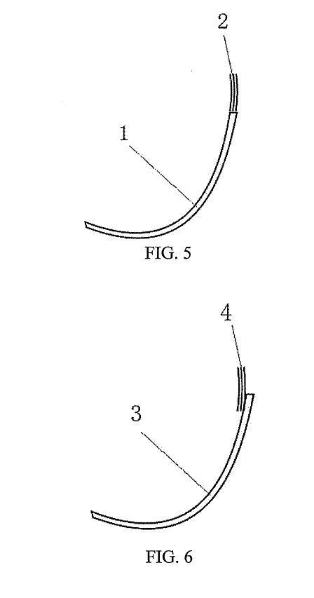

A medicated diaper capable of protecting the belly and navel of infants, wherein a protrusion structure is set in the front of the diaper to cover the belly and navel of infants, the protrusion structure 2 is the extending portion of the diaper body 1, it has printed or pressed pattern and has the mixed grains 9 of clove, cinnamon and long pepper. See FIGS. 1 and 5.

### IMPLEMENTATION EXAMPLE 2

A medicated diaper capable of protecting the belly and navel of infants, wherein a protrusion structure is set in the front of the diaper to cover the belly and navel of infants, the protrusion structure is a composite body made of the prolonged diaper surface and base film or absorbing material, or a composite body made of the prolonged diaper surface and base film plus absorbing material, the composite body has absorbent cotton dipped with liquid medicine for curing diarrhea and abdominal distention, the base film has a printed or pressed pattern and the absorbing material is colored.

### IMPLEMENTATION EXAMPLE 3

A medicated diaper capable of protecting the belly and navel of infants, wherein a protrusion structure is set in the front of the diaper to cover the belly and navel of infants, the protrusion structure 4 is a composite body of the extending portion of diaper body 3 and far-infrared material layer. The composite body has unguent 10 made of the mixture of clove, cinnamon and long pepper, the protrusion structure and the diaper are two separate parts combined into one structure by means of thermal sealing, adhesion or suturing. See FIGS. 2 and 6.

### IMPLEMENTATION EXAMPLE 4

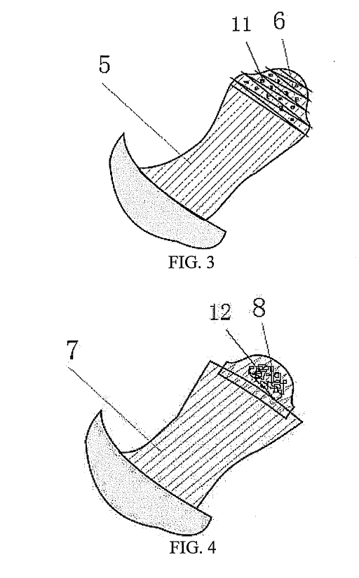

A diaper capable of protecting the belly and navel of infants, wherein a protrusion structure 6 is set in the front of the diaper 5 to cover the belly and navel of infants, the mixed grains 11 of clove, cinnamon and long pepper are provided on the protrusion structure. A protrusion structure 8 is arranged in the front of the diaper 7 to cover the belly and navel of infants, wherein the protrusion structure 8 is provided with absorbent cotton 12 dipped with liquid medicine made of cloves, cinnamon and long pepper. See FIGS. 3, 4 and 7. The protrusion is formed by the front of the diaper sealed and adhered to the structure protecting the belly and navel of infants (such as one material or composite body of several materials selected from regular non-woven fabric, special non-woven fabric, cast film, pure cotton material, fluff pulp and materials with far-infrared function). The diaper can not only protect the belly and navel of infants, but also effectively treat infant diseases such as diarrhea and abdominal distention.

What is claimed is:

1. A diaper, characterized in that a protrusion structure is set in the front of the diaper to cover the belly and navel of infants, wherein the protrusion structure is provided with caring medicament.

2. The diaper according to claim 1, characterized in that the protrusion structure is an extending portion of the diaper body.

3. The diaper according to claim 1, characterized in that the protrusion structure is a composite body made of a prolonged diaper surface and base film or absorbing material, or a composite body made of the prolonged diaper surface and base film plus absorbing material.

4. The diaper according to claim 1, characterized in that the protrusion structure is made of pure cotton fabric or regular non-woven fabric.

5. The diaper according to claim 1, characterized in that the protrusion structure has a printed or pressed pattern.

6. The diaper according to claim 1, characterized in that the protrusion structure and the diaper are two separate parts combined into one structure through connection.

7. The diaper according to claim 6, characterized in that the protrusion structure is connected with the diaper by means of thermal sealing, adhesion or suturing.

8. The diaper according to any one of claims 1 to 7, characterized in that caring medicament is provided inside or outside the protrusion structure, the said medicament is in the form of powder, grain, liquid for spraying, paste or absorbent cotton dipped with liquid medicine.

9. The diaper according to any one of claims 1 to 7, characterized in that the caring medicament can be one or the mixture of two or three selected from cloves, cinnamon and long pepper.
